(19) United States
(12) Patent Application Publication
(10) Pub. No.: US 2026/0013785 A1
(43) Pub. Date: Jan. 15, 2026

(54) Apparatus, Systems and Methods for Precise Operations on Subjects

(71) Applicant: Manish D Kulkarni, Pleasanton, CA (US)

(72) Inventors: James Ogg, Sunnyvale, CA (US); Manish Kulkarni, Pleasanton, CA (US)

(73) Assignee: Netra Systems, Inc, Pleasanton, CA (US)

(21) Appl. No.: 19/269,022

(22) Filed: Jul. 14, 2025

Related U.S. Application Data
(60) Provisional application No. 63/670,125, filed on Jul. 12, 2024.

(51) Int. Cl. A61B 5/00; A61B 3/10; A61B 3/18

(52) U.S. Cl. CPC A61B 5/445; A61B 3/102; A61B 3/18; A61B 5/0066; A61B 5/0071; A61B 5/0088; A61B 5/4547; A61B 2503/40

(57) ABSTRACT

We are presenting a system, and an apparatus for fluorescence imaging, detection and spectroscopy. The device also comprises OCT (Optical Coherence Tomography) imaging. The device can be used for diagnosis, imaging, measurements, evaluation or therapy. The device can be used for ophthalmic imaging and/or diagnosis, or retinal imaging and/or diagnosis. The apparatus and system can be used for the measurement and imaging of the eyes of humans as well as the animals.

### CROSS REFERENCE TO RELATED APPLICATIONS

The instant application is a non-provisional application and claims priority to provisional US patent application number. 63/670,125 titled “Apparatus, Systems and Methods for Precise Operations on Subjects” filed on Jul. 12, 2024. This benefit is claimed under 35. U. S. C. $119 and the entire disclosure of the Provisional U.S. patent Application Nos. 63/670,125, is incorporated here by reference for all of their teachings.

### FIELD OF TECHNOLOGY

The following description relates to a system, and an apparatus for fluorescence imaging, detection and spectroscopy. The device also comprises OCT (Optical Coherence Tomography) imaging. The device can be used for diagnosis, imaging, measurements, evaluation or therapy. The device can be used for ophthalmic imaging and/or diagnosis, or retinal imaging and/or diagnosis. The apparatus and system can be used for the measurement and imaging of the eyes of humans as well as the animals. The device can also be used for measurement/detection of other body parts of humans or animals. The device can also be used for measurement/detection of other living or non-living specimens.

### SUMMARY

The invention discloses a novel imaging/spectroscopy system, and apparatus for ophthalmology. The apparatus and system can be used for diagnosis, evaluation or therapy. The device can be used for ophthalmic imaging and/or diagnosis, anterior segment imaging and/or diagnosis, and/or retinal imaging and/or diagnosis. The apparatus and system can be used for the eyes of humans as well as animals. The device can also be used for the measurement/detection of other body parts of humans or animals. The device can also be used for the measurement/detection of other living or non-living specimens.

In an embodiment, a multi-modal system that can perform optical coherence tomography (OCT), scanning laser ophthalmoscopy (SLO) (fluorescence and reflectance), adaptive optics enhancement, and OCT-angiography simultaneously is presented. Such a system minimizes registration errors in multi-modality images, and accelerates biomedical research. Such a system could also permit enhanced high-speed clinical diagnosis for animals as well as humans.

In a few embodiments, this system would also permit a wide-selection of excitation wavelengths to measure fluorescence intensity from a variety of fluorophores and auto-fluorescing tissues.

### BRIEF DESCRIPTION OF THE DRAWINGS

FIG. 1 is a block diagram of the system modules.

FIG. 2 is an optical schematic of the positionable imaging head.

FIG. 3 shows the orbit arm, with the imaging head mounted.

FIG. 4 shows the system shuttle configured to support the orbit arm and the support cart.

FIG. 5 shows the removable workbench rigidly mounted to the shuttle, with the small animal positioner

FIG. 6 shows a detailed view of an adjustable mouse cartridge.

FIG. 7 shows a selection of small animal cartridges.

FIG. 8 shows a configuration where the eye's pupil (801 and 802) matches with the exit pupil of the objective

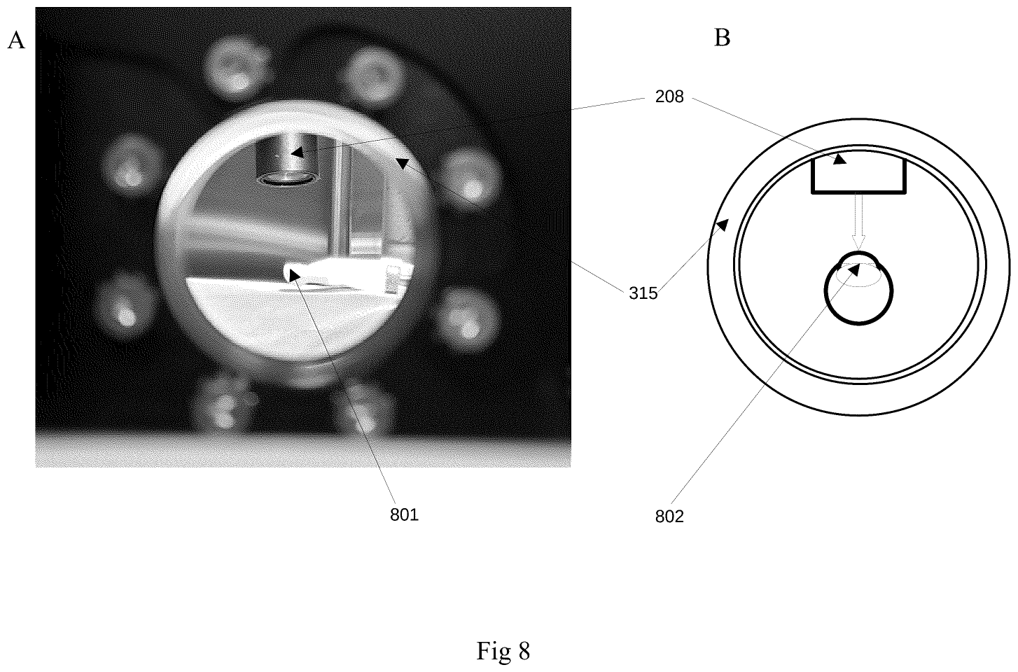

FIG. 9 shows the imaging system pointing downwards to image a subject. The subject could be lying on a table.

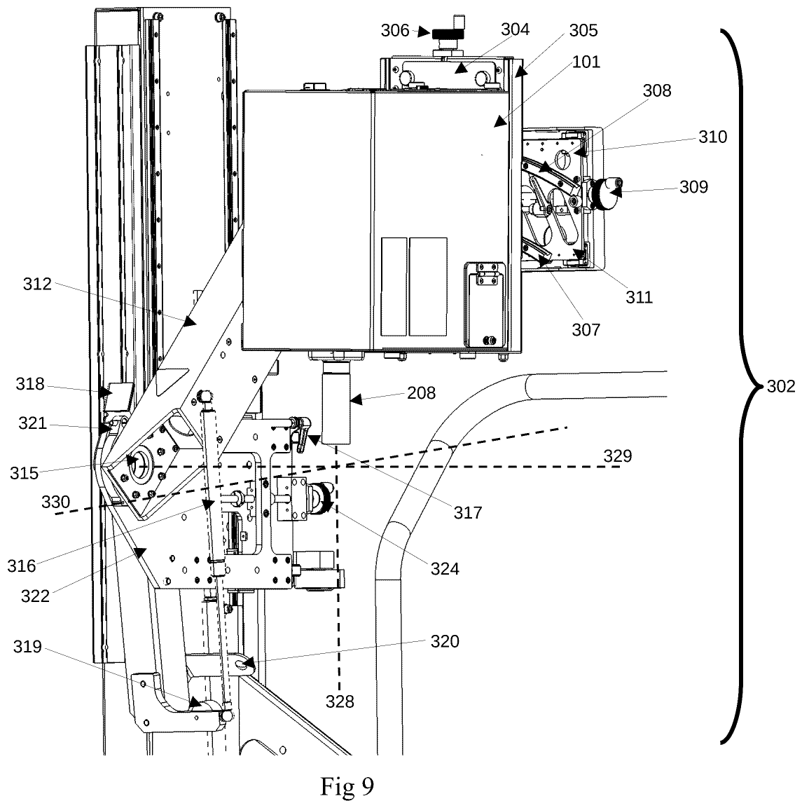

FIG. 10 shows the subject eye, with the pupil shown as a black disk. The X, Y, and Z axes are also shown. X axis bearing are shown.

FIG. 11 illustrates the moment arm.

FIG. 12 illustrated the rotation of the imaging head around the subject's eye.

FIG. 13 illustrates the subject eye and axes of rotation.

FIG. 14 illustrates a gimbal rotation causing an unwanted translation.

### DETAILED DESCRIPTION

The following describe the application of this system to imaging the eyes of research subjects.

The system can be used for diagnostics as well as therapy guidance.

This system could be useful in the differential diagnosis of skin diseases like inflammatory skin diseases (psoriasis, pemphigus). It could also be useful for skin aging, all skin cancers including non-melanoma skin cancer.

This system could be useful for Histopathological analysis of ex-vivo tissues.

Large subjects could include humans, non-human-primates, rabbits, pigs, rats, tree-shrews, mice, dogs, horses, goats, lambs, ships, etc.

In another embodiment the subject can include excised tissue, retinoid, organoid, stem-cells, non-living object under investigation, device or material under investigation.

This system could be used to diagnose and monitor the inflammatory skin diseases like psoriasis, pemphigus autoimmune skin diseases skin aging, skin cancers including non melanoma skin cancer, for differential diagnosis of autoimmune disorders of skin, or histopathological analysis of the ex vivo tissues.

A system for operating on a subject's organ under investigation, Where the system head rotates around the subject such that the center of rotation is a part of the organ under investigation of the subject. In some embodiments, the center of rotation is the center of gravity of the organ under investigation of the subject.

In some embodiments the organ is at least one of the eye, pupil, iris, tooth, teeth, mouth, cornea, skin, scalp a location on the skin or skin lesions.

In some embodiments, the operating procedure is at least one of ultrasound and optical imaging.

In some other embodiments, the optical imaging is at least one of scanning laser ophthalmoscopy, fundus camera, slit lamp, optical retinogram, electrical retinogram, fluorescence imaging, fluorescence life-time imaging, and optical coherence tomography.

In some embodiments, the optical imaging is at least one of scanning laser ophthalmoscopy, optical retinogram, optical spectroscopy, fluorescein angiography, optical coherence tomography, optical coherence tomography angiography, fluorescence lifetime imaging, depth resolved spectroscopy, optical coherence tomography spectroscopy, Doppler optical coherence tomography, adaptive optics, adaptive optics optical coherence tomography, adaptive scanning laser ophthalmoscopy.

In some embodiments, the operation is at least one of diagnosis and monitoring the Inflammatory skin diseases like psoriasis, pemphigus, autoimmune skin diseases, Skin aging, Skin cancers including Non melanoma skin cancer.

### Modular Imaging System

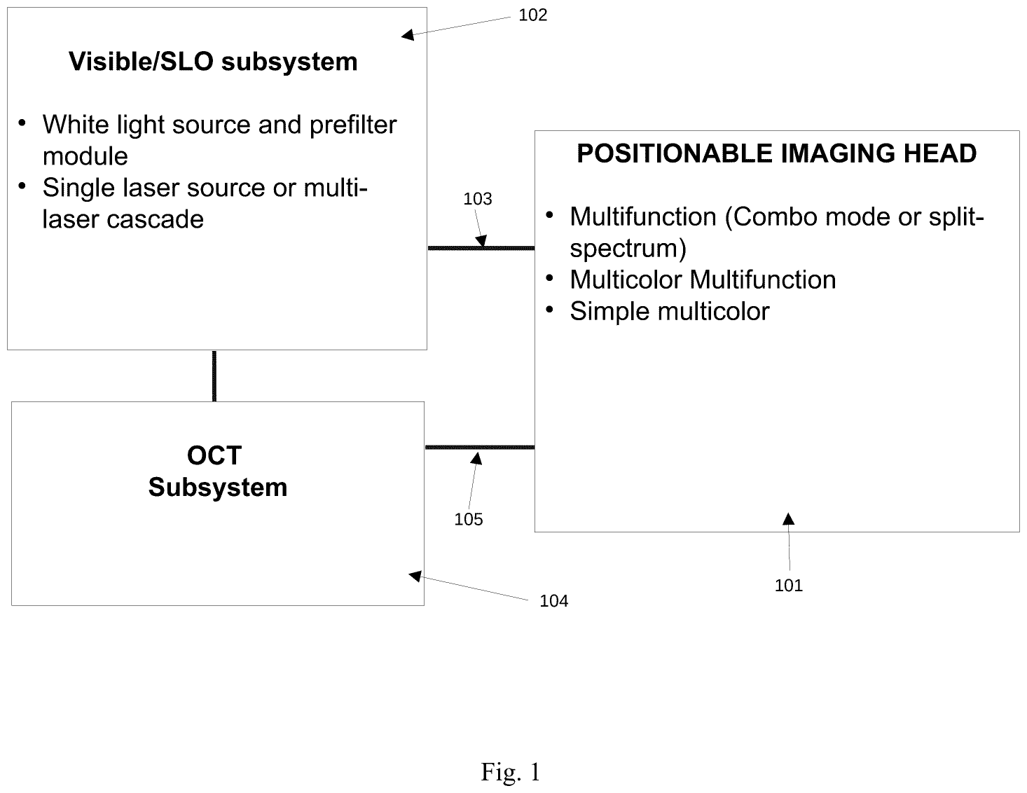

In a preferred embodiment shown in FIG. 1, a modular construction permits system flexibility. An imaging head 101 of minimal size can be positioned relative to the subject and can provide both SLO and OCT imaging.

A first group of modules 102 provides excitation for the SLO subsystem via a fiber optic cable 103. In one embodiment, this SLO module might be a broadband white light source and a prefilter module to eliminate wavelengths of light that are not well-handed by the filters inside the imaging head. The difficulty might come from the wavelengths being difficult to filter, or of high power which might be difficult to dissipate inside a small, enclosed space. In another embodiment, this SLO module might support one or more laser light sources, cascaded so as to provide light of one or more specific wavelengths. This cascading might be implemented via a fiber optic coupler) or by combining the beams in free space via dichroic mirrors. In another embodiment, the system might combine one or more narrow band or broadband light sources, each with independent control to permit rapid multiplexing between colors, etc.

In one embodiment, the SLO module might also receive and interpret fluorescence signals, and be connected via a first fiber optic cable of sufficient bandwidth to efficiently transmit both the excitation and fluorescent emission light wavelengths (e.g. S405-XP). In a preferred embodiment, the fluorescent emission light would be interpreted in the imaging head, permitting the imaging of fluorophores with longer emission wavelengths (e.g. Indocyanine green) than could be handled by a single optical fiber that could also transmit excitation for fluorophores with a shorter wavelength (e.g. green fluorescent protein).

A second module 104 provides input and handles the output for the OCT subsystem. In one embodiment, this module includes a cascade of superluminescent diodes serving as a broadband near-infrared light source, a reference arm, and a spectrometer, and is configured as a Michelson interferometer. A fiber coupler connects these three elements to the imaging head via a second fiber optic cable 105. In a preferred embodiment, the spectrometer and reference arm are mechanically isolated from the module's enclosure to provide protection against external shock and vibration. Also in a preferred embodiment, the reference arm provides a long, motorized stage for adjusting the reference arm length; a motorized neutral density filter disk that can be used to optimize the reference arm power; a means of controlling the polarization of the light returning from the reference arm; and optional glass added to match the optical dispersion of the glass in the sample arm.

Also in a preferred embodiment, the first fiber optic cable is selected to transmit visible light, and the second is selected to transmit near infrared radiation.

These first and second modules will connect to the positionable imaging head via fiber optic cables and electrical cables.

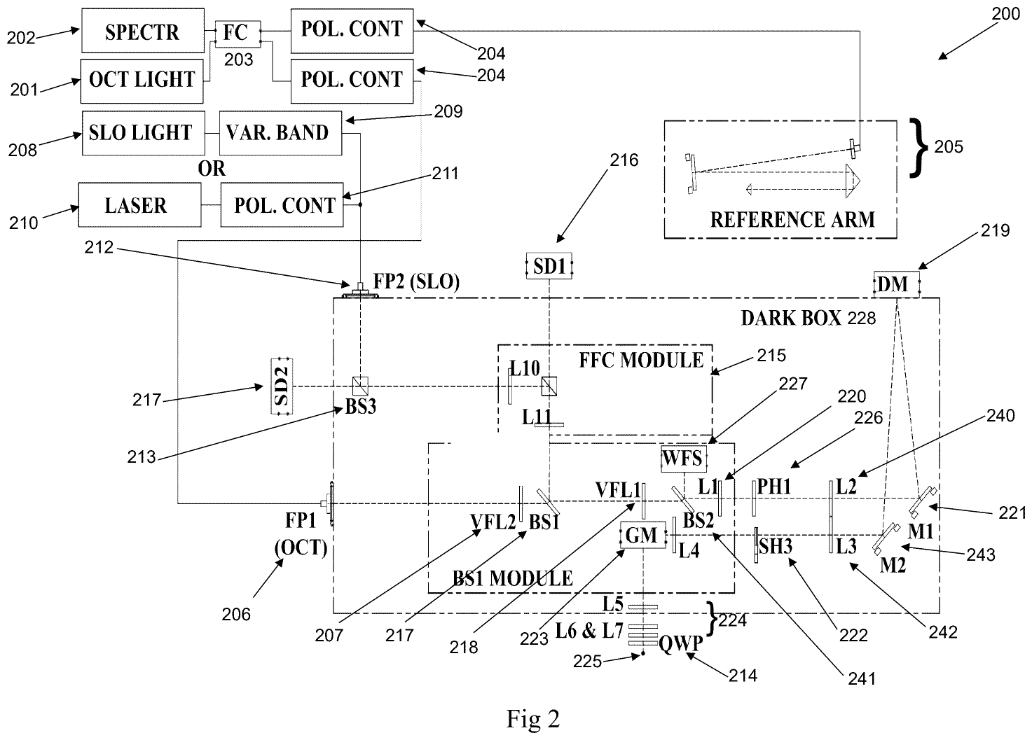

The system (FIG. 2) comprises of an OCT subsystem, the SLO subsystem, and optics common to both.

The OCT subsystem comprises a broadband OCT light source 201, such as a single superluminescent diode or an array of superluminescent diodes, and a spectrometer 202. In an embodiment, these are joined via a fiber coupler 203 to a pair of polarization controllers 204 and, to the OCT reference and sample arms. These polarization controllers 204 can be mounted so that they can be accessed and adjusted by the user during system operation. The reference arm 205 reflects the portion of the OCT excitation directed to it by the fiber coupler back to generate interference of the light. In embodiment, this arm includes a motorized or translating stage that can be used to set the distance from which the light is reflected back (reference arm path-length). The sample arm connects to the SLO/OCT-common-optics through an adjustable fiber port 206.

Additional glass or material of various types, to balance the optical dispersion of the reference and sample arms, can be added to the reference arm, the sample arm, or both. (Not shown.) Optical dispersion is matched in hardware by using at least 2 types of materials with different second and third order dispersion coefficients. For example, highly dispersive material such as SF11 and a moderate dispersive material such as BK7 could be used to match the dispersion in both the reference and sample arms.

An electrically-controlled-variable focus lens 207 (VFL2) adjusts the OCT sample arm light to compensate for chromatic aberrations due to wavelength differences between the OCT and SLO light. This variable focus lens can be controlled manually or automatically.

As some implementations of the system will operate on variable wavelengths, the chromatic aberrations can be expected to vary as a result.

The SLO subsystem is comprised of excitation, filtering, and detection. SLO excitation can be from a broadband (in some embodiments, the light can be visible, infrared or ultraviolet) light source 208, filtered through a variable bandpass filter 209.

In an alternate embodiment, SLO excitation can be provided by a fixed-wavelength laser 210 such as a 488 nm cyan laser, and an optional polarization controller 211. This light connects to the SLO/OCT common optics through an adjustable fiber port 212. It is then divided by a beam splitter 213. This beam splitter might reflect a certain ratio of the light, e.g. reflecting 70% and transmitting 30%. Alternatively, it might reflect or transmit light based on its polarity. This option might utilize a polarization controller either at the laser 211, or between the fiber port 212 and the beam splitter 213.

In one embodiment, a quarter wavelength plate 214 can be used to rotate the polarization of the light backscattered from the subject. This quarter wavelength plate 214 can be mounted to permit rotation by the user for adjustment through a machined crown, secured by a plastic-tipped setscrew or thumbscrew, or a rotational optical mount 503. The beam splitter 213 redirects the SLO excitation towards the fluorescence filter cube FFC module 215.

4 In one embodiment, the FFC module 215 uses a translating (optionally motorized) stage 301 to change between multiple fluorescence filter cubes 302. The FFC cubes typically contain an excitation filter, a dichroic, and an emission filter. Each cube would have filters selected for a specific fluorophore (i.e., fluorescence band) or sets of fluorophores.

In an embodiment, the motorized or translating FFC stage 301 positions the FFC cubes 302 so that the SLO excitation passes through the excitation filter and reflects off of the dichroic (such a dichroic is called longpass-dichroic-beamsplitter). The fluorescence filter cube module 215 may have lenses that focus the light onto the filters.

The light returning from the specimen again passes through the fluorescence filter cube module 215. Fluorescence passes through the long-pass-dichroic beamsplitter and emission filter in the fluorescence filter cube 302. This light will then be detected by the fluorescence detector 216, which might be a photo-multiplier tube. In turn, light reflected by the subject will be again reflected by the dichroic in the fluorescence filter cube 302, towards the reflectance detector 217, which might be an avalanche photodetector or a photomultiplier tube (PMT).

In some embodiments, a shortpass-dichroic-beamsplitter which would transmit the excitation light and reflect fluorescence emission light could be used.

4 In some embodiments, these detectors may have lenses to focus the light upon them, and shutters to block light during service. These shutters might be mechanically controlled, for example, to prevent exposure of sensitive optical elements to light when the system is powered down and the cover removed for service.

In some embodiments detectors may be fully or partially con-focal, proceeded by a focusing element and pinhole, with size equal to or larger than the diffraction limit.

The excitation from OCT and SLO subsystems are combined by a beam splitter 217 in the integrated beam splitter module 501. This beam splitter 217 can be a single element.

In an embodiment, this beam splitter 217 can be selected from an array of optical elements installed on a translating or motorized stage 502 that permits various choices of 217 to be selected from a longwave pass dichroic, a shortwave pass dichroic, or a mirror. The various choices for 217 would permit various imaging modes. A longwave pass dichroic will pass the longer wavelength band allocated to the OCT (e.g. NIR) and reflect shorter wavelength band allocated to the SLO (e.g. visible light). This mode permits the combined operation of both SLO and OCT (This is the commonly used imaging mode).

In another embodiment, the shortwave pass dichroic 217 permits OCT to operate in a shorter wavelength band, with the fluorescence due to the OCT excitation occurring in an allocated longer wavelength band. This mode is called split-spectrum mode. For example, it might be used with fluorophores such as ICG (Indocyanine Green), allocating one shorter wavelength NIR band for OCT and fluorescence excitation, and one longer wavelength NIR band for SLO fluorescence detection.

In an embodiment, a mirror can be chosen for element 217 to permit SLO operation over wavelength ranges overlapping with the OCT source light. This would be an SLO only imaging mode.

The combined OCT and SLO excitation, depending on the mode, pass through an electrically-controlled variable focus lens 218. This VFL can be used to electronically make small adjustments in the focal length of the system optics. It can also be utilized to automate the optimization of focal length, and reference arm length and the working distance (the distance between the objective and the subject) in the sample arm. This combined beam then is directed to a (typically electrically) deformable mirror 219.

In an alternate embodiment, the VFL1 218 could be placed in a path exclusive to SLO (anywhere in the light-path-between element 217 BS1 and the fiber port FP2). 207 (VFL2) can be used to focus the OCT sample arm light at a depth location of interest. That way the SLO and OCT can be focused anywhere independently per the operator's choice. In an embodiment, the operator could use VFL 218 to focus the SLO image in the same focal plane as the focused OCT en-face image. Thus chromatic aberrations (difference in focal length at different wavelengths of light used for OCT and SLO) could be used by independently controlling the SLO and OCT images using independent VFLs.

In an embodiment, the beam out of the VFL 218 passes through a telescope comprised of two achromatic lenses 220 and 240 to expand the beam as it and is directed towards the deformable mirror 219 by a beam-folding mirror 221. The beam is then directed by a second folding mirror 243 and reduced by a telescope comprised of two achromatic lenses 241 and 222.

This deformable mirror 219 permits adaptive optics by changing shape to compensate for optical aberrations in the subject and elsewhere in the system. These deformable mirrors can be paired with adapters so that they can be interchanged without major modification or realignment to the system. In another embodiment, a flat mirror could be substituted for a less expensive system without adaptive optics.

An optional emergency shutter 222 blocks excitation in cases of system failure or in response to an emergency stop switch. In an embodiment, this shutter has a rapid electrical control, and might monitor the galvo control signal, so as to prevent a localized area of the eye from being exposed to high-power light for too long if the galvos stop.

This beam is then reflected by single or dual axis scanning mirrors 223. Many scan patterns are possible for example, a raster pattern, a line scan, an XY scan, a circle scan, radial line scans, etc. An objective 224 then causes the beam to converge toward a moving spot on the subject 225. OCT and SLO reflectance, and SLO fluorescence, will follow the optical path back. A pinhole 226 blocks stray light.

The deformable mirror 219 can be adjusted using software programs to try to optimize the resulting images.

Alternatively, some of the returning light can be diverted by a beam splitter to a wavefront sensor 227 to perform sensor-based adaptive optics.

In an embodiment, the common optics of the system are housed in a dark box 228 to eliminate stray light. To permit airflow while limiting light intrusion, this dark box 228 can be equipped with dark vents 601 that have curved internal passages that permit some airflow while blocking direct light leakage. These dark vents 601 can share mounting features with modular commercial cable pass-throughs 602, so that either cable routing or airflow routing could be configured without needing to modify the dark box walls.

In other embodiments, the SLO reflectance detection is in the SLO module. In this configuration, the SLO fiber would ensure confocality, so an additional spatial filter might not be needed. In still another embodiment, the SLO reflectance detection is omitted, and reflectance information provided by OCT.

This imaging head could either be handheld or mounted to a frame. This frame might support a mechanism to position the imaging head, hold the imaging head in a fixed position relative to a mechanism to position the subject, or have dual mechanisms to position both the imaging head and the subject.

This frame might also house the supporting modules such as the computer, OCT support module, and/or SLO modules. Alternatively, the computer and other support modules can be mounted on a secondary cart, which might be positioned next to the imaging head during procedures such as imaging, surgery, etc.

To isolate the imaging head from vibration from the computer, etc., it may be necessary to have two rigid frames. In one embodiment, the upper frame might secure the imaging head rigidly to a mechanism that positions the subject. Vibration isolation mounts would connect this upper frame to a lower frame that would house the support modules.

In another embodiment, the mechanism to position the imaging head might connect to the primary frame.

### Orbit Arm for Imaging Head Positioning

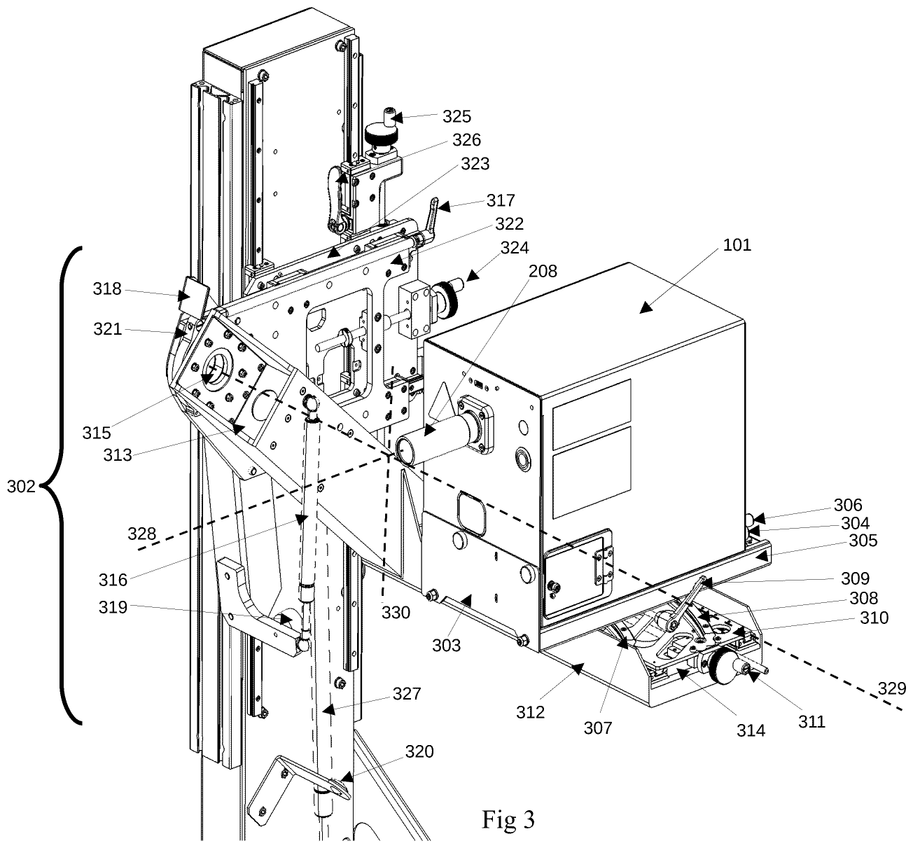

FIG. 3 shows a positioning arm 4.02 that can be used to precisely translate and rotate the mounted imaging head 1.01 relative to the subject. This permits precisely interacting with (or operating on) large subjects. This imaging head can be used for test, measurement, imaging or therapeutics. The test & measurement can be electronic (e.g., ERG: Electro-retinogram) or optical or ultrasound. The imaging method can be optical or electronic or ultrasonic, including optical coherence tomography (OCT), scanning laser ophthalmoscope (SLO), adaptive optics imaging (AO), OCT-angiography, fundus imaging, etc. Diagnostics and therapeutics could include retinal and corneal surgeries, laser surgeries, anterior-chamber surgeries, cataract surgeries, intraocular lens surgeries.

Operators will often want to rotate from imaging or scanning or aiming at one area of the subject's retina (or fundus or eye or another organ, e.g., teeth, a spot on the subject's body or the skin) to another area. It is often difficult or impossible to precisely adjust the angle of the subject's eye, or organ, relative to the body. For small animal subjects, it is practical to rotate the head, or head and body, of the subject. For larger subjects, it is more practical to rotate the imaging head.

Ideally, this rotation should be centered near the pupil or iris of the eye when the operator is performing retina or fundus imaging. In another embodiment the rotation should be centered on the organ to be operated on, e.g., teeth, or a location on the skin, or mouth, etc. Rotation around any other point would be equivalent to a rotation around the pupil or iris or the organ of interest, plus an unwanted translation. This translation would be proportional to the distance between the actual center of rotation and the pupil or iris or the organ and/or tissue of interest. After a slight rotation, the operator would have to move the head back, to eliminate the unwanted rotation. Since the imaging head's field of view might be very small, this unwanted rotation might result in the loss of the image being used to adjust the position, complicating the process.

To provide space for the subject or the head of the subject, the imaging head arm and joints need to be away from the head of the subject or the superficial lesions of the organs of the subject or organs of the subject, and away from any support or fixturing under the subject. While the virtual axes of rotation should pass through the subject's eye, the mechanism implementing the axes will need to be offset away from the subject.

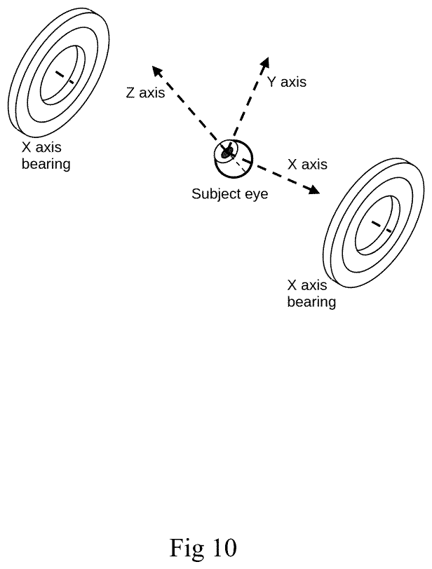

In FIG. 10 shows the subject eye, with the pupil shown as a black disk. The X, Y, and Z axes are also shown. The subject's head, surrounding the eye, is not shown. An orbital camera motion is desired. That is, the camera should rotate around the subject, specifically the subject's pupil. Two rotational bearings are shown. The physical axes of these bearings are also shown, as a short dashed line. To provide clearance for the head of the subject, these bearings need to be away from the eye. However, to permit rotation around an axis through the pupil of the eye, the physical axes of these bearings must be coaxial with the axis through the pupil. This common axis needs to be a virtual axis. The mechanism defining the axes need to be away from the subject.

This offset can be along the virtual axis as shown (eg 315). It can also be radial, with the bearings have a large radius (eg 307, 308).

Many types of bearings have a hole in them that is coaxial to their axis of rotation. This hole can be used as a visual indication of said axis. Operators would be able to align one axis of the eye to the bearings by translating the eye until the eye's pupil was lined up with the hole in the bearing.

The center of rotation of the imaging head is different than its center of gravity

The goal is to rotate the imaging head around the pupil of the eye without translation.

The system has mechanisms Xo, Yo, Zo to position the “node” of the imaging objective. A “node” is defined as the point of intersection of the scanning beams. It is also known as “exit pupil”. Positioning the exit pupil of the objective becomes primarily important when the system can be used with various objectives, that have different fields of views, lateral resolutions and different exit pupil diameters. These are the system translations.

Translation axes: X′, Y′, Z′ allow placing the intersection of the axis of rotation at the subject pupil. The goal is to match the exit pupil of the system to the subject pupil and match these to the intersection of all 3 (X, Y, Z) axes of rotation. These are the subject translations.

X′, Y′ Z′; change the position of the subject relative to the axes of rotation.

Xo, Yo, Zo are changing the position the node of the objective at the intersection of the axes of rotation. Zo becomes useful when we change between the objectives.

Rotating around the camera's center of gravity is easier for engineers, but not useful for operators.

In an embodiment, the rotating around the pupil of the eye is preferred.

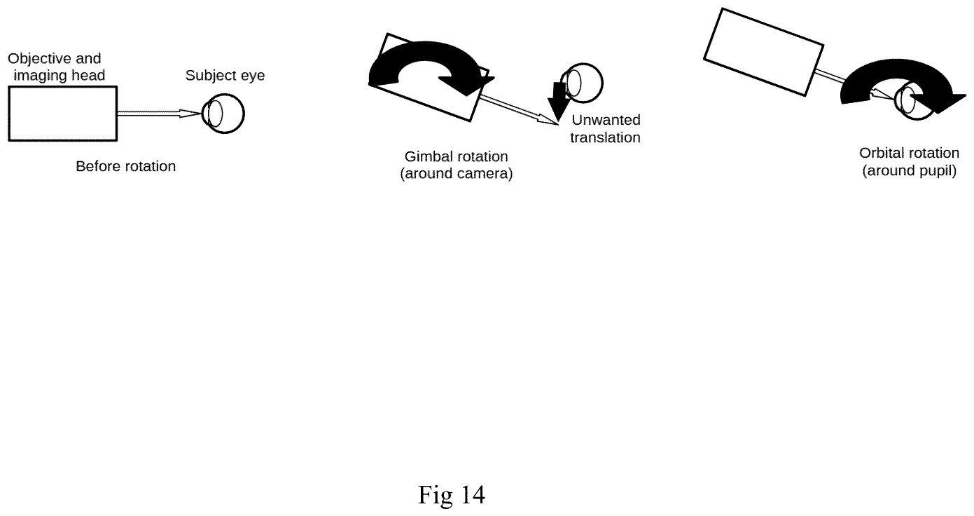

In an embodiment, the optical axis of the eye is coaxial with the optical axis of the objective on the imaging head (FIG. 14). To the extent that all lenses in the objective and subject are each symmetric around their own axis, this assumes that the lenses are aligned to a common axis, can be called the Z axis. Most in vivo imaging subjects will have an opaque iris blocking light around a clear pupil. The middle point of the subject's pupil can be called the eye node. The X and Y axes are mutually perpendicular to the Z axis.

Each objective will have a characteristic node along its axis. This node might be where the scan pattern converges to a point. Conceptually, this node and the axis define the pupil plane. This can be thought of the objective's exit pupil. In an embodiment, it is where the subject's pupil should be for best imaging. This objective is designed to work with the lens of a typical subject's eye.

In practice, any subject will have an eye that is a little different, and so the optimal Z distance might vary slightly. Some eyes might be significantly different, and so require substantial deviation from this idealized case.

In an embodiment, for retinal or fundus or the eye's posterior chamber imaging, the objective's axis should pass through the eye's node, and the objective's node should be close to the eye's node. When this is the case, the scanning pattern from the objective is at its narrowest as it passes through the most restrictive aperture of the eye. This permits beams with a larger NA (for a higher transverse resolution) and a larger scan pattern.

However, rotation is generally necessary. Often, the subject or imaging head will need to be rotated to line up their optical axes. Additionally, operators might want to view the eye from an angle that differs from the subject's optical axis. For example, they might want a perspective that is more nasal, more temporal, more superior, or more inferior. The objective's node and the eye's node should be matched, even if their pupil planes are not parallel.

Many cameras will rotate around a point close to their own center of gravity (FIG. 14). This minimizes the force necessary to rotate them or hold them in place. This rotation can be called a gimbal rotation. Implementing a gimbal rotation is usually not difficult mechanically. However, it makes imaging the subject difficult. Every rotation will come with an unwanted translation (FIG. 14). System operators might need to guess how much to rotate, then rotate by that amount. This will cause an unwanted translation. They will generally lose the image (e.g., OCT image) of the retina (or fundus) due to the translation. They will need to eliminate the unwanted translation by translating back, possibly manually. If they have completed these maneuvers correctly, they might have reacquired the image of the retina. If not, they might not have any visual feedback about what mistake was made. If their estimated rotation was too large, too small, or about the wrong axis, operators will need to repeat each step of the process.

For these reasons, it is preferable to rotate while keeping the objective node at the eye node (FIG. 14). This is accomplished by rotating around the nodes. This can be called an orbital rotation. Because orbital rotation avoids unwanted translations, operators might be able to maintain a view of the retina while rotating. As a result, they will maintain visual feedback of the retina while navigating. This greatly reduces the risk of wrongly estimating an adjustment and not being able to recover the image.

This rotation is mechanically more difficult. The objective node and the eye node need to be at the axes of rotation. This state of the imaging head, objective, and subject, can be called centered. If a system can use objectives of different lengths. centering a system generally involves two sets of translations. One set of translations might be used to position the eye node of each subject at the axes of rotation. This set of translations will adjust for the individual variations between subjects. This set can be called the subject translations, the primes, or X′, Y′, and Z′. Another set of translations might be used to adjust for any differences in the system, such as when changing between objectives of different lengths. This set can be called the system translations, the nots, or Xo, Yo, and Zo. In some implementations, these translations might be controlled by design. For example, Xo and Yo might be set during assembly, and operators only using Zo when switching between objectives. A fully adjustable positioner for orbital rotation will have eight degrees of freedom. These include two axes of rotation, around X and Y. Rotation around Z might be accomplished simply by rotating the output in software. These also include two sets of three translational degrees of freedom. Xo, Yo, and Zo are used to move the objective's node to the rotational axes. This adjustment might not be needed often. X′, Y′, and Z′ are used to move the node of the subject's eye to the rotational axes. This adjustment might be needed for each different subject.

In addition to requiring up to eight degrees of freedom, orbital rotation is more mechanically difficult for two additional reasons: First, the rotation needs to be constrained by bearings. These bearings must rotate around the eye, but must not interfere with the eye or other parts of the subject. Second, the imaging head's center of gravity will not be near the axes of rotation. As a result, it might tend to rotate downward. Depending on the weight of the imaging head, the forces involved might be large enough to cause injury if not ameliorated mechanically.

In some embodiments, bearings need to be placed away from the center of rotation, otherwise they will interfere with the subject.

In some embodiments, we need to offset the torque of the camera mechanism and need to try to rotate around the subject eye and lower its center of gravity, need some mechanism to adjust the center of gravity of the head, degrees of mechanisms need to be supported else we risk a falling head.

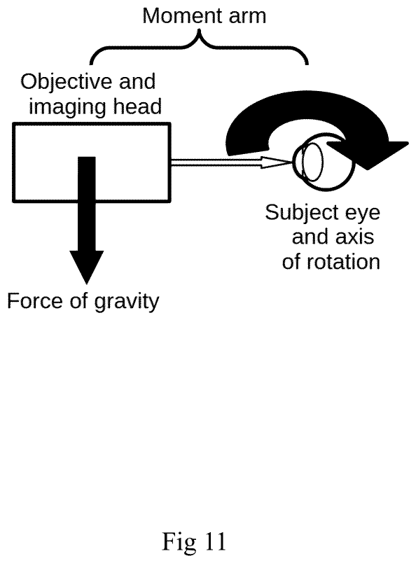

Because our imaging head rotates around axes that are away from its center of gravity, it will tend to rotate downwards. The magnitude of this torque will be equal to the force of gravity, times the perpendicular distance between the vector of the force of gravity and the axis of rotation. This is the moment arm (FIG. 11).

Many camera systems avoid significant torques from gravity by configuring the camera to rotate around axes that are close to its center of gravity (ie gimbals). This results in a small moment arm, and a small torque.

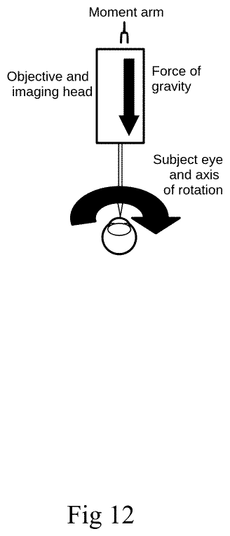

It is useful to rotate the imaging head around the subject's eye (FIG. 12). As a result, the torques from gravity need to be offset. This could be accomplished using a counterweight. This counterweight would move the overall center of gravity towards the center of rotation. However, this might mean having mechanism on both sides of the subject. This might not be practical, for example, when the subject is laying on a table.

This could also be accomplished using force sources, such as gas springs, to offset gravitational forces. These will need to be carefully engineered. When the imaging head is horizontal, the force source will need to offset the full weight of the imaging head. However, when the imaging head moves toward and then past vertical, the moment arm becomes shorter, and can actually change direction. To offset this, the force source will also need to transition from pushing strongly, to pushing weakly or even pulling weakly.

Rotation of the head could cause it to fall, the center of gravity of the head is in the middle of the head. Head needs to be supported when we do the rotations.

In one embodiment, the center of the hole 315 (the center of the bearing X rotation bearing) is aligned with the subject's eye. This helps position the subject eye at the centers of the rotation.

Implementation bearings permit rotation around X axis. Can be used as a visual indication of that X (one) axis of rotation.

This offset rotation can be achieved by circular bearings, offset along their axis of rotation. e.g., they might be above or beside the subject's head. For rigidity, it might be necessary to have multiple sets of ball bearings, roller bearings, etc. These would be on the same or on different sides of the subject's head or lesions, on a common axis.

This offset rotation could also be achieved by using curved rails. These would be similar to linear translation rails, but would be curved around the axis of rotation so that a carriage mounted to the rail would rotate as it traveled along the rail. A uniform rotation could be achieved using a rail of a constant radius. For rigidity, it might be necessary to use multiple curved rails. These rails would need to be installed such that their axes of rotation are the same. For example, curved rails of different radii could be mounted in a common plane, such that the curved rails are coaxial. Alternatively, curved rails of the same radius could be mounted to different planes. These planes would need to be perpendicular to the common axis of rotation. Alternatively, a similar motion could be implemented using sets of rollers that roll against a curved surface. The rollers and surface might have a V-shaped or similarly non-flat contact area to hold the rollers in-plane with the surface. Alternatively, it might be a three-dimensional curved surface (e.g. a sphere) to provide three axes of rotation.

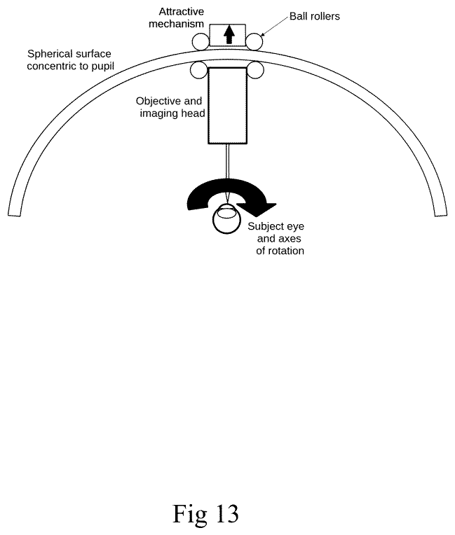

FIG. 13 is a sketch of this concept. Instead of using a large-radius bearing for one rotation on a first rotational stage, and a perpendicular large-radius bearing for a second rotation on a second rotational stage, a spherical surface could be used. This spherical surface would be concentric to the axes of rotation (e.g. the pupil). The imaging head would roll along this spherical surface on ball rollers. A separate attractive mechanism might work on the opposite side of the spherical surface, forcing the imaging head against the surface. This attraction might be mechanical if the angles of rotation are limited. Alternatively, this attraction might be magnetic. Alternatively, the spherical surface might be made from a magnetic material, and magnets mounted to the imaging head to pull it against the spherical surface.

This offset rotation can also be achieved by the use of multiple rotational bearings on multiple rigid members (e.g. a four bar mechanism).

To avoid the need for large, manual forces from the operator, the rotation and translation forces might need to be internalized. Vertical movements can be offset using conventional springs, constant-force springs, gas springs, etc. These avoid the need for the added mass of counterweights, and in the case of vertical rotations, the need for a weighted arm on the opposite side of the subject, or a second weighted arm that is connected to the first via gearing. These springs might pivot with the in-plane rotation, to increase or decrease the applied torque to match that of the imaging head and arm, which would change as the moment arm rotates relative to the direction of gravity.

In a preferred embodiment, the orbit arm mechanism will provide two axes of rotation, mutually perpendicular to the axis of the objective. It will also provide three degrees of freedom for translating the imaging head towards the point of intersection of the axes. Finally, it will provide a primary system translation along the axis of the objective.

In this embodiment, the imaging head 101 is secured to the first system translation stage 305 by a front bracket 303 and a back bracket 304. These brackets are slotted to provide for travel in a second and third direction, if necessary to center the imaging head relative to the rotations.

(The Z axis is defined as the axis of the objective. The X axis is defined as the horizontal axis perpendicular to Z. The Y axis is mutually perpendicular to X and Z. The Y axis might be vertical, diagonal, or horizontal.)

The first system translation stage is actuated by a crank 306. This stage translates along the axis of the interchangeable objective 208 to provide the correct working distance for the objective in use.

This stage in turn mounts to the Y rotation stage. This stage uses two constant-radius rails, one with a small radius 307 and one with a large radius 308, both concentric to the Y axis. Rotation along these rails is prevented by tightening the Y rotation brake, actuated by a lever 309. The rotation Y stage mounts to the first subject translation stage 310 that runs on linear rails, actuated by the crank 311.

The first subject translation stage mounts to the orbit arm weldment 312. The weldment has shear walls 313 and 314 that limit deflection. Specifically, they are engineered to reduce the change in deflection as the arm is rotated around the X axis e.g. from having the horizontal objective to having a vertical objective. The orbit arm weldment mounts to a circular bearing 315 that permits rotation around the X axis. The length of the arm weldment offsets this bearing along its axis relative to the subject. The torque around the X axis caused by the weight of the imaging head is partially offset by the rotation around the gas spring 316. This gas spring is mounted such that it tilts as the arm rotates around X. This alters the torque applied by the gas spring around the X axis to more closely match the torque from the imaging head, which similarly varies with rotation around X.

Rotation of the arm weldment around X is controlled using a clamp 321 that is tightened using a remote lever 317. This rotation is limited by upwards travel stop 318, relative downward stop 319, and absolute downwards travel stop 320.

The X rotation bearing mounts to the second subject translation stage 322 which is, in turn, mounted to the third subject translation stage 323. These translations are adjusted with cranks 324 and 325, respectively. The second subject translation stage, being long and vertical, has a lock 326 that can be used to bypass the crank 325 for large movements, and a gas spring 327 to offset the weight of the orbit arm and imaging head.

### Mobile Shuttle

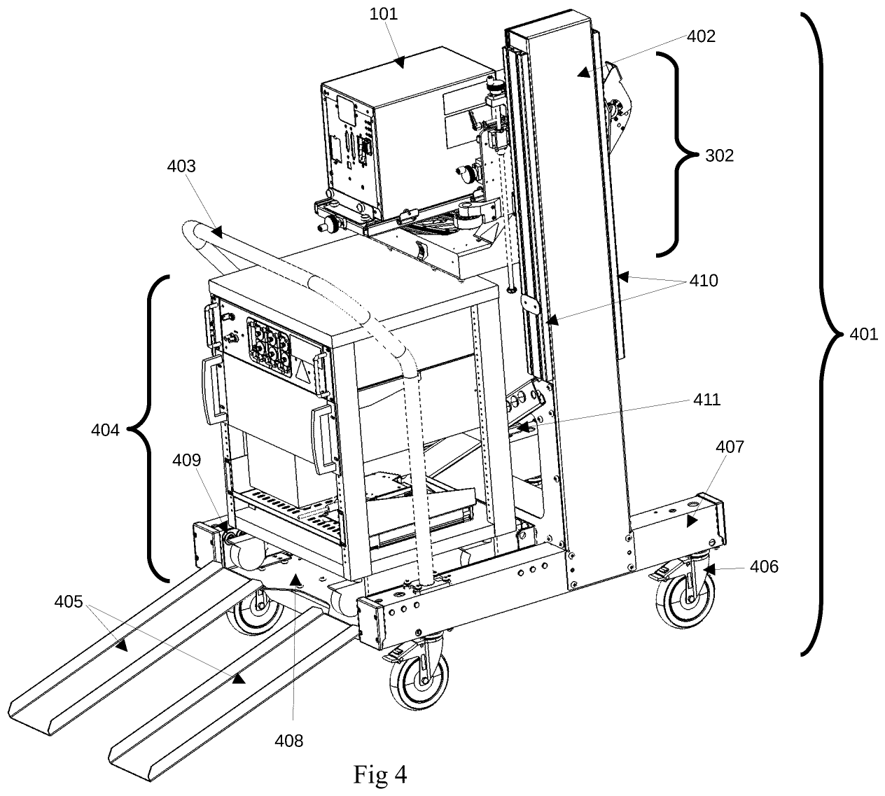

FIG. 4 shows the imaging head 101 and orbit arm 302 mounted to a mobile shuttle 401. This shuttle would consist of a frame that has casters 406, so that it can be transported e.g. from one lab to another.

For ease of transport, the shuttle may provide a bay 408 that the support cart 404 can be loaded into. This support cart will house the computer, SLO module, OCT module, etc. This bay can have elastomer mounts 409 to isolate the imaging head from shocks as the cart is loaded and unloaded, and from vibration from the computer, etc. The elastomer mounts will also provide an additional layer of shock isolation for the modules on the support cart during transport from one location to another. Removable ramps 405 can be used to load the support cart onto the bay. These ramps can be either two (one under each set of cart casters) or one unified ramp.

The shuttle might provide rubber lead-in structures to help to locate the support cart as it is loaded on, as well as rubber catches and/or straps to secure the cart in place.

The shuttle casters 406 might be solid (e.g. solid rubber) or pneumatic for better isolation from shock & vibration. Alternatively, they might be combined castors and leveling feet. They might be mounted directly to the legs 407, or via hinged mechanisms to reduce shocks from forward motion. The axis of the hinges can be above the casters, to be more rigid to vertical forces (e.g. up-and-down vibration) yet more flexible in response to transverse forces (e.g., from a crossing a raised threshold perpendicular to the direction of travel).

The shuttle might be equipped with T-slotted extrusion e.g. 410 on which accessories can be mounted.

In a preferred embodiment, the shuttle will also provide a push handle 403. This push handle might be one unified piece (e.g. bent tubing), two separate posts, etc.

This embodiment can be configured without the push handle and cart bay 408 to minimize the system footprint, providing better access to the imaging head and/or subject.

In an alternative configuration, the handle and cart bay can be omitted if the shuttle will not be moved often.

### Accessory Workbench

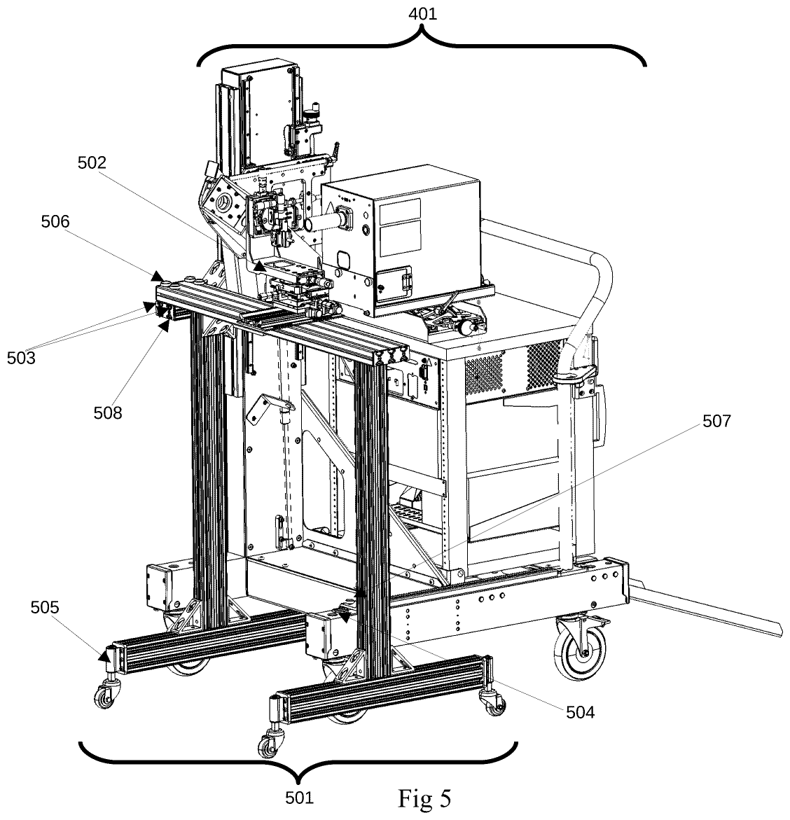

FIG. 5 shows an accessory workbench 501 could be mounted to the shuttle to permit one imaging head to be used with both a small animal positioner 502 for small animals, and with an orbit arm for large animal. In one embodiment, this workbench would mount directly to and be supported by shuttle 401. This bench could be simply screwed to nuts in the T-slot extrusion 410. In another embodiment, the workbench could fold or rotate into place, and then fold or rotate away when not in use.

In a preferred embodiment, this workbench would roll on casters 505 that would float on springs. To install the workbench, it would be rolled towards the shuttle. Bars with lead-in 503 and 504 would slide into the T-slot extrusion on the shuttle leg 411 and on the support beam 508 which is secured to the T-slot extrusion on the shuttle post 410. Then they would be secured by tightening thumbscrews 506 and 507.

Either embodiment of this workbench would hold the small animal positioner 502 when in use. Other accessories e.g. a warmer controller, etc., could also be mounted to the workbench cart.

To image large animals, the workbench could be unmounted and moved out of the way.

### Cartridges

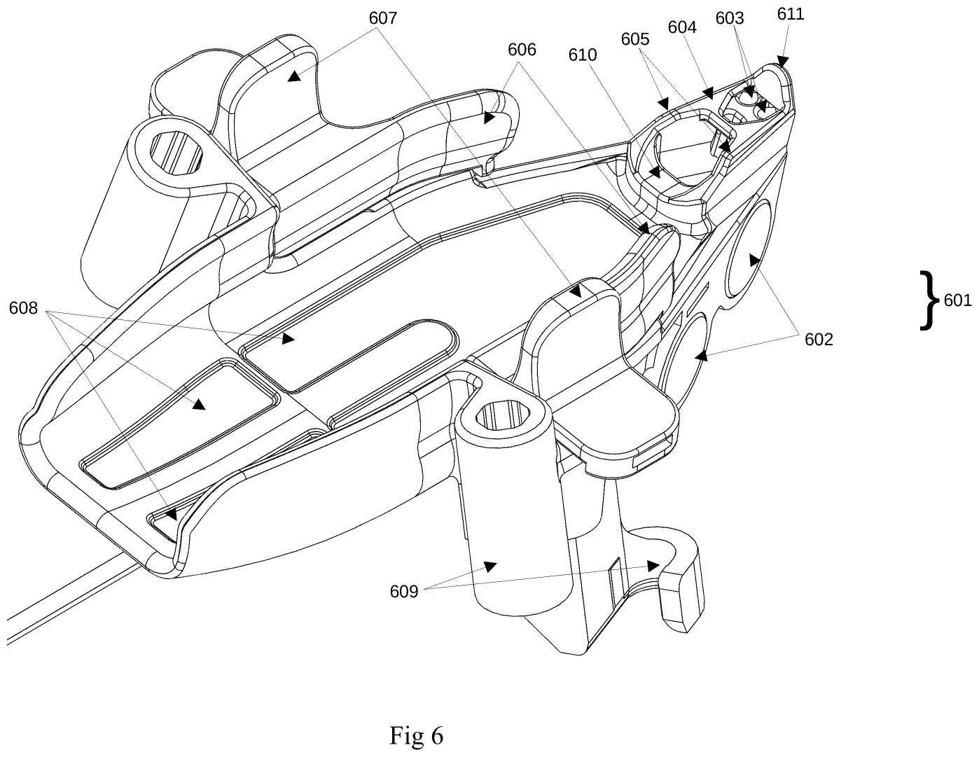

Cartridges are mounted on the small animal positioner to house small animals such as mice, rats, pinoles, lizards, anole lizards, dunnart, chickens. tree-shrews, etc. FIG. 6 shows a detailed view of a preferred embodiment of a mouse cartridge 601. Primary refinements offered by this embodiment are the magnetic fasteners for a strap that prevents the mouse's head from tilting 602 and adjustable side supports 606. Cartridge features, especially for small subjects, might be proportionately small and so be awkward to handle. These fasteners might be permanent magnets (e.g. rare earth magnets) mounted to the cartridge. The head straps can be made of rubber with iron oxide particles, woven from martensitic stainless steel (e.g. 410 or 440c alloy) wire, etc. The straps can be engaged by wrapping around the subject and placed in contact with the magnet, and disengaged by pulling away from the magnet. This could be an involute motion, ensuring a constant tension on the subject. The magnets can be sized as to be strong enough to support the small subject's weight, yet weak enough to be unable to sustain an excessive force on the small subject.

In this preferred embodiment, the adjustable side supports 606 are also secured magnetically. They permit one cartridge to be widened or narrowed to adjust to specific subjects, reducing the number of sizes of cartridge needed. The adjustment of each side is independent, so that users who primarily operate on (or image) one eye (e.g. the right eye) can support the downhill side of the subject. The uphill side can be away from the subject, providing more room to insert and remove the subject. Adjustment of the side supports can be made easier by providing small handles 607 that can be gripped e.g. with a thumb and forefinger and slid in or out against the small friction due to the magnetic forces.

For marginally heavier subjects, teeth could be added between the cartridge and side supports, and arranged so that they interlock to oppose the weight of the subject. These teeth might also restrict rotation. For example, two sets of teeth on either end of the side support would permit one thumbscrew to secure the location and rotation, without needing to be very tight. The teeth can also be away from the subject, while the thumbscrew is near the subject, under a curved section. When tilted, the weight of the e.g. rat or tree shrew will be on the curved section, engaging the teeth without significant load on the thumbscrew.

In another embodiment, the near fastener on each side is comprised of a magnet and a magnetic material.

To repeatably register the mouse, the cartridge has dual mounting features 609 that position the cartridge in a precise location to present the mouse at a specific position and angle of tilt e.g. to initially direct the optical axis or the optic nerve towards the objective for imaging.

These mounting features would be located relative to a bite bar 604 that uses the mouse's incisors to ensure a precise location of the mouse's head, a palate bar that uses the mouse's incisors and molars, etc.

The cartridge has two passages 603 that run through the cartridge and connect to tubes under the cartridge, one can be used to gaseous anesthesia (e.g. isoflurane) to the subject's nose. The other is used to remove waste gases. Walls 605 on either side of the bite bar and a raised ridge under the subject's jaw 610 limit the leakage of waste gases.

The area above the bite bar, including the mouse's nose, can be enclosed by a flexible strap or a rigid hood. For the subject's safety, the flexible strap must wrap around a front wall 611, offset from the subject's nose, as well as around bony areas of the subject's muzzle. This ensures that the subject's nose is not compressed. This strap will secure to the cartridge e.g., using the magnetic fasteners 602.

The cartridge may include depressions 608 that will prevent drainage of small amounts of fluids (e.g. urine) expelled by the subject.

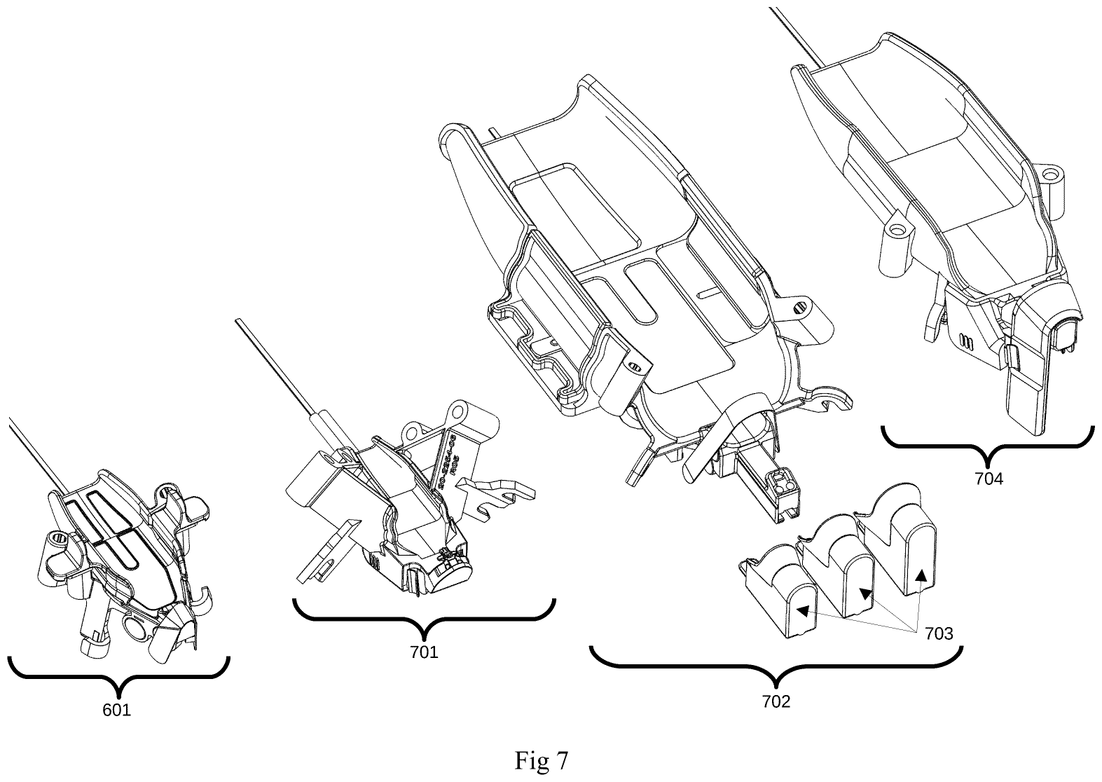

FIG. 7 shows a selection of small animal cartridges. On the left, there is an adjustable mouse cartridge 601, oriented towards the optic nerve. Next, there is an alternative construction of mouse cartridge 701, oriented towards the optic disk. Next there is a rat cartridge 702 with hoods in multiple sizes 703 for use with gaseous anesthesia. These hoods could include a conic section, roughly matched to the size and shape of the subject's muzzle. Depending on the subject, multiple sizes or adjustability might be needed. The hood will also need a section with volume around the subject's nose, to ensure that it is not compressed. The hood might be made of transparent material to permit operators to confirm visually that the subject's nose is not compressed. The hood should either have sufficient adjustment to disengage to permit placement and removal of the subject, or the hood should be easy to remove and replace.

A cartridge for tree shrews 704, oriented towards their optical axis is shown on the right of FIG. 7.

1. A system for operating on a subject's organ under investigation,
where the system head rotates around the subject such that the center of rotation is a part of the organ under investigation of the subject.
The system of claim 1 where the center of rotation is the center of gravity of the organ under investigation of the subject.
The system of claim 1 wherein the organ is at least one of the eye, pupil, iris, tooth, teeth, mouth, cornea, skin, scalp a location on skin or skin lesions.
The system of claim 1 wherein the operating procedure is at least one of ultrasound and optical imaging
The system of claim 1 wherein the optical imaging is at least one of scanning laser ophthalmoscopy, optical retinogram, optical spectroscopy, fluorescein angiography, optical coherence tomography, optical coherence tomography angiography, fluorescence lifetime imaging, depth resolved spectroscopy, optical coherence tomography spectroscopy, Doppler optical coherence tomography, adaptive optics, adaptive optics optical coherence tomography, adaptive scanning laser ophthalmoscopy.
The system of claim 1 where the operation is at least one of diagnosis and monitoring the inflammatory skin diseases like psoriasis, pemphigus, autoimmune skin diseases, skin aging, skin cancers including Non melanoma skin cancer
A system for operating on a subject's organ under investigation,
Where the system head rotates around the subject such that the center of rotation is a part of the organ under investigation of the subject,
wherein an accessory workbench could be mounted to permit one imaging head to be used with both a small animal positioner for small animals, and with an orbit arm for large animals.
A system for optical imaging of the retina, wherein the exit pupil of the system to the subject pupil and match these to the intersection of all 3 (X, Y, Z) axes of rotation.
